(19) United States
(12) Patent Application Publication
(10) Pub. No.: US 2008/0242975 A1
(43) Pub. Date: Oct. 2, 2008

(54) DEVICE AND METHOD FOR LOCATING A CANNULA THAT IS INSERTED INTO A BODY

(75) Inventors: Ban C.H. TSUI, Edmonton (CA); Heinrich PAJUNK, Geisingen (DE); Horst PAJUNK, Geisingen (DE)

Correspondence Address: AKERMAN SENTERFITT, P.O. BOX 3188, WEST PALM BEACH, FL, 33402-3188, US

(73) Assignee: PAJUNK GMBH & CO. KG BESITZVERWALTUNG, Geisingen (DE)

(21) Appl. No.: 12/055,885

(22) Filed: Mar. 26, 2008

(30) Foreign Application Priority Data
Mar. 29, 2007 (DE) 10 2007 015 553.2

(51) Int. Cl. A61B 5/06; A61N 5/067

(52) U.S. Cl. 600/424; 607/89

(57) ABSTRACT

A light pointer (16) is mounted on the proximal end of a cannula (10) by use of an adapter (14) for positioning the cannula (10) that is inserted into a body by use of an ultrasonic head. The light pointer (16) emits a light beam (18) in a direction axially parallel to the cannula (10). When the cannula (10) is inserted, the light beam (18) produces a light spot on the bodily surface on which the ultrasonic head is placed.

### TECHNICAL FIELD

This application relates to a device and a method for positioning a cannula that is inserted into a body.

### BACKGROUND

In medical practice cannulas are inserted into the human body for various purposes. Such cannulas are used in particular in anesthesia, e.g. in plexus and spinal anesthesia. In this regard it is extremely important to locate and track the position of the cannula, in particular the cannula tip, in the body. To this end it is known to place on the bodily surface an ultrasonic head by means of which the cannula may be positioned.

In practice, the ultrasonic head is generally placed by hand. Difficulties may arise in positioning the cannula when the ultrasonic head is placed in such a way that its visual field does not detect the insertion channel of the cannula.

### SUMMARY

One object is to provide a device and a method for positioning a cannula that is inserted into the body, using an ultrasonic head by means of which it may be reliably ensured that the ultrasonic image records the cannula penetrating the body.

This object is achieved according to the devices and methods having the features disclosed herein.

According to one embodiment, a light pointer, preferably a laser pointer, which emits a light beam in the distal direction in a plane encompassing the longitudinal axis of the cannula is placed on the cannula. The light beam preferably extends essentially axially parallel to the cannula. When the cannula is inserted, the light pointer produces a light spot on the bodily surface in the distal direction in front of the puncture site. This light spot indicates the location at which the hand-guided ultrasonic head must be placed. Since the light beam and the cannula lie in the same axial plane, it is ensured that the ultrasonic visual field of the ultrasonic head detects the cannula axis and, therefore, the cannula penetrating the body.

The light pointer, in particular in the form of a laser pointer as frequently used in presentations, as a key chain attachment, and the like, is an economical article. The light pointer is preferably detachably mounted on the cannula, in particular on a proximal projection of the cannula, by use of an adapter. This allows the light pointer to be used multiple times in an economical manner.

The light spot produced by the light pointer may have any given shape, and in the simplest case has the shape of a point of light. It is advantageous for the light spot to have a two-dimensional shape with structures extending in different directions. The shape of a cross, for example, is practical, wherein one arm of the cross extends in the direction of the cannula and a second arm of the cross extends transversely, preferably perpendicularly, to the direction of the cannula.

This design of the light spot also increases the target accuracy when placing the ultrasonic head. The two-dimensional, in particular cross-shaped, design of the light spot also allows a rotation of the cannula together with the light pointer placed thereon to be easily recognized. If the plane defined by the light beam and the cannula axis is perpendicular to the bodily surface, a light spot is produced which is symmetrical with respect to this plane. If the defined plane is inclined relative to the bodily surface, this results in an asymmetrical distortion of the light spot. In this manner it can be ensured that the plane defined by the light beam from the light pointer and the cannula extends perpendicular to the bodily surface, and therefore that the ultrasonic head is precisely placed in this plane.

### BRIEF DESCRIPTION OF THE DRAWINGS

The devices and methods are explained in greater detail below with reference to one exemplary embodiment illustrated in the drawings, which show the following:

FIG. 1 shows a side view of a cannula with a light pointer placed thereon; and

FIG. 2 shows the geometric relationships upon insertion of the cannula.

### DETAILED DESCRIPTION

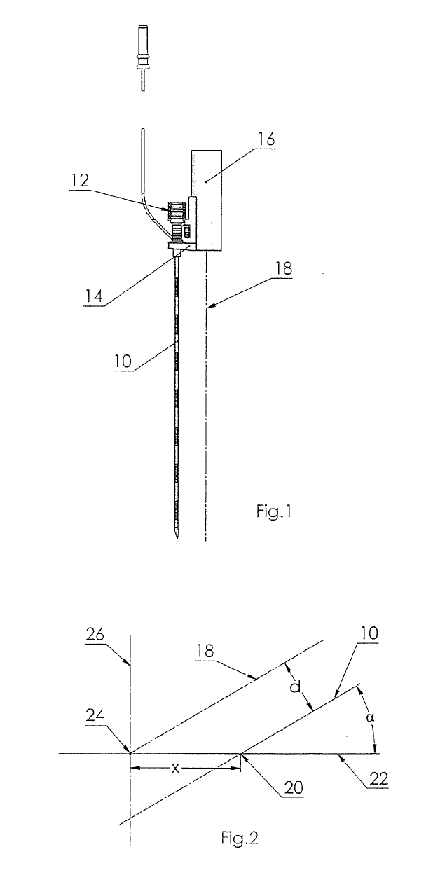

FIG. 1 shows the device according to one embodiment connected to a standard cannula, e.g. a Tuohy cannula.

The cannula 10 has a projection 12 at its proximal end. An adapter 14 is detachably fastened to this projection 12. The adapter 14 bears a light pointer 16 which in particular is designed as a laser pointer. Such laser pointers are generally known in the prior art, and therefore a detailed description is unnecessary. In particular, such a laser pointer has a battery-powered laser diode which emits light in the visible wavelength range. Red laser diodes are preferred for cost reasons.

The adapter 14 is preferably made of plastic, and may be clamped onto the projection 12 of the cannula 10, e.g. by spring-loaded clamping jaws or a screw clamp. The adapter 14 may be fixedly connected to the light pointer 16.

The light pointer 16 emits a light beam 18, which in FIG. 1 is illustrated as a dashed-dotted line. The light beam 18 extends axially parallel to the center axis of the cannula 10. The light pointer 16 is preferably designed using an exit aperture in such a way that the light beam 18 produces a cross-shaped light spot, one arm of which extends in the plane defined by the light beam 18 and the cannula 19, and the other arm of which extends perpendicular to this plane.

As schematically shown in FIG. 2, the cannula 10 is inserted at an angle α into the bodily surface 22 of the patient at the puncture site 20. The cannula 10 together with the light pointer 16 is guided such that the plane defined by the cannula axis and the light beam 18 (in FIG. 2, the plane of the drawing) is perpendicular to the plane of the bodily surface. The light pointer 16 is attached to the cannula in such a way that the light beam 18 emitted from the light pointer 16 is at a distance d from the cannula axis. The light beam 18 correspondingly produces a light spot at a point 24 which in the distal direction is at a distance x in front of the puncture site 20 of the cannula 10. The distance x is d/sin α. The flatter the angle at which the cannula 10 is inserted, the greater the distance x. A distance d between the cannula 10 and the light beam 18 of d=17 mm, e.g., results in distances x between the puncture point 20 and the light spot 24 of approximately 31 mm for a puncture angle α=30°, approximately 42 mm for a puncture angle α=20°, and approximately 65 mm for a puncture angle α=15°.

A hand-guided ultrasonic head known as such (not illustrated in the drawing) is placed on the point 24 of the light spot, e.g. on the intersection point of the cross-shaped light spot, wherein the axis 26 of the visual field of the ultrasonic head, represented by a dashed-dotted line in FIG. 2, extends perpendicular to the bodily surface 22. This ensures that the visual field of the ultrasonic head detects the cannula 10 when it penetrates the body along the dashed-dotted line shown in FIG. 2.

1. A device for positioning a cannula (10) inserted into a body by use of an ultrasonic head which may be placed on the bodily surface (22), comprising:
a light pointer (16) proximally situated on the cannula (10) which emits a light beam (18) in a distal direction in a plane encompassing a longitudinal axis of the cannula (10) and produces a light spot (24) on the bodily surface (22) in a distal direction in front of a puncture site (20) of the cannula (10) for placement of the ultrasonic head.

2. The device according to claim 1, wherein the light pointer (16) is a laser pointer.

3. The device according to claim 1, wherein the light pointer (16) is detachably mounted on a proximal projection (12) of the cannula (10) by use of an adapter (14).

4. The device according to claim 1, wherein the light beam (18) from the light pointer (16) extends axially parallel to the longitudinal axis of the cannula (10).

5. The device according to claim 1, wherein the light spot (24) has a two-dimensional shape.

6. The device according to claim 5, wherein the light spot (24) has the shape of a cross, in particular having an arm which intersects an axis of the cannula (10) and an arm which extends perpendicular thereto.

7. A method for positioning a cannula inserted into a body by use of an ultrasonic head which may be placed on the bodily surface, comprising the steps of:
directing a light pointer mounted on the cannula to emit a light beam that extends in a distal direction and in a plane that encompasses a longitudinal axis of the cannula;
inserting the cannula into the bodily surface at an angle a such that the light beam produces a light spot on the bodily surface in a distal direction in front of a puncture site of the cannula,
placing the ultrasonic head on the bodily surface at the light spot.

8. The method according to claim 7, wherein the light pointer is mounted on the cannula such that the light beam from the light pointer extends axially parallel to the longitudinal axis of the cannula.

9. The method according to claim 7, wherein the light pointer produces a light spot on the bodily surface which is formed in two dimensions, and upon insertion of the cannula, further comprising the step of rotating the cannula about its longitudinal axis in such that a plane defined by the light beam from the light pointer and the cannula is oriented perpendicular to the bodily surface, and the light spot is symmetrical with respect to this plane.

10. The method according to claim 9, wherein light spot is formed in the shape of a cross.
